(19) United States
(12) Patent Application Publication
(10) Pub. No.: US 2014/0329770 A1
(43) Pub. Date: Nov. 6, 2014

(54) Compositions Comprising Phytoestrogens Receptor Beta Agonists and Dietary Fibers for Chemopreventive Treatment of Sporadic Adenomatous Polyposis

(71) Applicant: CM&D PHARMA LIMITED, Esher, Surrey (GB)

(72) Inventors: Maria Cristina Comelli, Padova (IT); Alfredo Di Leo, Monopoli (IT)

(21) Appl. No.: 14/347,507

(22) PCT Filed: Sep. 24, 2012
(86) PCT No.: PCT/EP12/03990

§ 371 Date: Mar. 26, 2014

Related U.S. Application Data
(60) Provisional application No. 61/542,127, filed on Sep. 30, 2011.

(51) Int. Cl. A61K 31/717; A61K 31/357

(52) U.S. Cl. CPC A61K 31/717; A61K 31/357
USPC 514/57

(57) ABSTRACT

It is herein discloses compositions comprising a blend of dietary phytoestrogens receptor β agonists and dietary fibers for prevention and treatment of sporadic adenomatous polyposis. The aforementioned compositions proved to be effect in a clinical trial enrolling patients prone to polyp recurrence and at intermediate risk for colorectal cancer (CRC) development.

### FIELD OF INVENTION

The present invention relates to compositions comprising one or more oestrogen receptor β agonist phytoestrogens in association with lignans and dietary fibres, insoluble and resistant to enzymatic digestion, and to the use of said compositions for prevention and treatment of intestinal mucosa and in particular of Sporadic Adenomatous Polyposis (SAP) and Familial Adenomatous Polyposis (FAP).

### FIELD OF THE INVENTION

Early initiating events in non-inheritable human colorectal tumorigenesis include the somatic mutation of the tumour suppressor gene Adenomatous Polyposis Coli (APC) [Vogelstein B et al. 1988], making intestinal cells susceptible to further tumour promotion and progression through the accumulation of epigenetic mutations, e.g. hypermethylations [Duthie S J, 2011]. This occurrence is responsible for the progressive silencing of transcriptional programmes functionally related to the apoptotic regulation of epithelial cell cycle in the colon. Lifestyle behaviors, particularly western-style diets high in pro-oxidants, fat, refined sugars and potentially carcinogenetic components facilitate epigenetic modifications of apoptosis programmes [Lund E K et al., 2011]. Despite promising results from dietary intervention studies in animals and colon neoplastic cell lineages, and epidemiological studies linking dietary phytochemicals and fibre consumption with a reduced risk for colorectal cancer (CRC) development [Gingras D et al., 2011], the efficacy of dietary chemoprevention of human established tumorigenesis and recurrent neoproliferation conveyed so far conflicting results. Sporadic Adenomatous Polyposis (SAP) is a human APC mutated, non-inheritable form of intestinal neoplasia due to an unbalanced proliferation to apoptosis ratio in colon epithelial cell cycle. Through the adenoma-adenocarcinoma sequence pathway, the intestinal neoplasia can progress to CRC. Estrogens have been reported to exert a protective role against CRC development [Woodson K et al., 2001]. In the adenomatous tissue of SAP and Familial Adenomatous Polyposis (FAP) patients, the decreasing ERβ expression is a surrogate biomarker of prognostic value for patient's progression along the adenoma-adenocarcinoma sequence [Bardin A. et al., 2004].

The inventors previously demonstrated that the non-adenomatous mucosa (“normal appearing”) of the Apc^{Min/+} mice, fed with a high fat diet to induce intestinal neoplasia, differentiated from the intestinal lining of the healthy wild congener fed the same diet, because of a decreased ERβ and decreased apoptotic biomarkers such as TUNEL and caspase-3. Supplementing the Apc^{Min/+} mice with selected dietary ERβ agonists and non-starch, insoluble and indigestible fibers, in particular those qualified for a 6% lignin content, completely recovered ERβ to the healthy wild level in the non-adenomatous mucosa, at the same time increasing its expression in the adenomatous tissue [Di Leo and Barone M, 2009; Barone M. et al., 2010]. Thereby, a significantly reduced development of intestinal neoplasia was found, as per a 34% decreased polyp number and dysplasia in the small intestine and colon. If confirmed in the human non-adenomatous mucosa of patients with established and recurrent intestinal neoplasia, these evidences could be prodromic to the secondary chemoprevention of polyp recurrence and progression to advanced adenomas in patients presenting with established intestinal neoplasia, and to the primary chemoprevention in the general population, presenting with known risk factors for intestinal neoplasia development.

Nowadays, a growing number of SAP patients potentially targeted by CRC screening programs are prescribed chronic low-dose aspirin as a tool for secondary chemoprevention, because of the reported reduced polyp recurrence and risk for CRC [Chan A T et al., 2012]. Nonetheless, concerns on long-term side effects require clinical monitoring, and long-term, safe secondary chemoprevention of the adenoma-carcinoma sequence remains of scientific interest, and the object of active development. Recently, a large ACF-based, CRC chemopreventive, prospective and randomized trial on a 6-months intervention with sulindac, atovarstastin and a proprietary prebiotic blend (ORAFTI® Synergy 1) in subjects at increased risk for CRC did not demonstrate significant reductions in rectal ACF numbers, when compared with maltodextrins. Notably, none of the interventions had a significant effect on cellular proliferation, whereas a similarly increased pro-apoptotic effect was observed in active and placebo groups, making their chemopreventive potential indeterminate in sporadic CRC. However, no assessment of basic dietary intake and/or habits was provided [Limburg P J et al., 2011].

Therefore, chemopreventive treatment or therapeutic treatment of Sporadic Adenomatous Polyposis (SAP) or Familial Adenomatous Polyposis (FAP) are a need still felt.

### SUMMARY OF THE INVENTION

In a randomized, double blind and placebo controlled study the inventors studied the impact of supplementing a composition for oral administration comprising a blend of ERβ-targeted dietary phytoestrogens and non-starch, insoluble and indigestible fibers qualified for lignin content to which it is further added lignans, on ERβ, TUNEL, caspase-3, ERα, and Ki-67 expression in the non-adenomatous mucosa of SAP patients prone to polyp recurrence, actively ongoing the surveillance program by screening colonoscopy every 3-5 years and classified at intermediate risk for CRC development because of the histological assessment of polyp dysplasia from previous polipectomies.

Therefore, in a first aspect an object of the present invention are compositions comprising at least one ERβ-targeted phytoestrogens in combination with non-starch, insoluble and indigestible dietary fibers qualified for having a content not less than 5% lignin content, and lignans qualified for having a content of secoisolariciresinoldiglucoside (SDG) not less than 40%.

Yet, it is a further object of the invention an use of the compositions according to the present invention for prevention and treatment of Sporadic Adenomatous Polyposis (SAP) and Familial Adenomatous Polyposis (FAP) and preferably the use of these compositions is for the secondary chemopreventive treatment of Sporadic Adenomatous Polyposis (SAP) and Familial Adenomatous Polyposis (FAP).

### BRIEF DESCRIPTION OF THE FIGURE

FIG. 1. Study design and flow chart of the clinical trial on sporadic adenomatous polyposis (SAP) patients.

### DETAILED DESCRIPTION OF THE INVENTION

In SAP and FAP, the progressive ERβ silencing is involved in the silencing of apoptosis, and it is a prognostic factor in the promotion and progression of the adenoma-carcinoma sequence. Cells skipping apoptosis migrate more slowly along the crypt-villus axis and are less efficiently cleared into the intestinal lumen, finally leading to polyp formation. Therefore, polyp development and recurrence represent the macroscopical findings of an altered epithelial turnover. The surveillance program by screening colonoscopy is the main step to detect the adenoma-carcinoma pathway, to surgically remove suspicious/dysplastic polyps, and to follow-up for polyp recurrence and progression to advanced stages. Recurrence is often thought to reflect the growth of “missed polyps”, too small or proximally located to be detected and removed during previous screening colonoscopies, or regrown because of piecemeal removal. The inventors hypothesized a ERα prevailing and/or ERβ decreased expression in the non-adenomatous mucosa of SAP patients prone to polyp recurrence, to pave the way for an altered apoptotic control of cell proliferation.

These patients present an intermediate risk for colorectal cancer (CRC) development and are the privileged target for chemopreventive treatment of established intestinal neoplasia by suitable dietary supplementations.

The compositions according to the present invention comprising at least one ERβ-targeted dietary phytoestrogens in combination with non-starch, insoluble and indigestible fibers qualified for having not less than 5% lignin content and lignans qualified for at least 40% of secoisolariciresinoldiglucoside (SDG). The addition of the lignans in association with phytoestrogens and lignin is functional to the need to make early available the same to the intestinal mucosa for the time needed to the lignin to be intestinally converted in enterolignans by the human intestinal microbiota.

It is a further requirement of the present invention that each ingredient is blended into the composition in a lower dose than the one otherwise needed for the single component to similarly exert the desired effect, thus leading to synergistic and/or potentiating effect, with the added advantage of higher safety, even over long-term exposure.

For the purpose of the present invention the phytoestrogens are selected from the group consisting of silymarin, silybinin, iso-silybinin, silydianin, silychristin and mixtures thereof and preferably the ERβ-targeted phytoestrogens is the Milk Thistle extract consisting of the mixture of silymarin, silybinin, iso-silybinin, silydianin, silychristin and more preferably the Milk Thistle extract titered at not less than 70% silymarin and 30% silybinin.

As for the non-starch insoluble and indigestible fibres qualified for having at least 5% lignin content, preferably these fibres further comprise cellulose and hemicellulose, and are selected from extracts of cereals, such as but not limited to oat, wheat bran, wheat grains, barley, or extracts from legumes, such as but not limited to pea fibers.

The lignans are selected among those able to be directly detected as such into human urine, or through their intestinal metabolites enterodiol and enterolactone, and are preferably vegetable extracts titered at least in 40% SDG and more preferably are selected from extracts of flaxseed, sesame seeds, rapeseeds, soy flour, whole-grain cereals (wheat, oats, rye and rice) and coffee beans.

In addition for the present invention the components of the compositions are in a ratio by weight of at least of:
1:4:0.1 (dietary phytoestrogens:non-starch insoluble and indigestible fibers:lignans as SDG) when referred to dietary phytoestrogens and lignans in extracts;
1:6:0.2 when referred to the dietary phytoestrogen silymarin:non starch insoluble and indigestible fibers:lignan as SDG; and
1:14:0.4 when referred to the dietary phytoestrogen silybinin:non starch insoluble and indigestible fibers:lignans as SDG.

The subjects eligible for this treatment are SAP and FAP patients ongoing the surveillance program by screening colonoscopy for the follow-up of adenoma (polyp) recurrence and progression to adenocarcinoma, irrespective of being found recurrent or non-recurrent at surveillance screening colonoscopy, and with their non-adenomatous mucosa evolving to develop intestinal adenomas. The treatment is addressed to oppose the new development of intestinal adenomas, and their progression to advanced forms, favoring a correct epithelial turnover to occur via a balanced proliferative to apoptotic ratio of colonocyte cell cycle, by mean of a ERβ increased expression on the intestinal mucosa and thereby activating the related transcriptional programs, particularly the ones related to the apoptotic control of cell growth.

Therefore, it is a further object of the invention the use of the compositions of the invention for the secondary chemoprevention of recurrence and progression in SAP and FAP patients, in a subject at risk for intestinal neoplasia occurrence on the basis of his/her predisposing factors, such as familiarity, genotype, lifestyles, dietary habits and age.

In order to assess whether the compositions object of the inventions are effective for prevention and treatment of SAP and FAP a clinical study has been carried out in SAP patients prone to polyp recurrence focusing on the behavior of the non-adenomatous mucosa following exposure to the compositions of this invention.

Sixty patients naïve from previous and concomitant CRC chemoprevention were randomized to placebo or active supplementation with a composition according to the invention (herein identified as ADI and composed by 175 mg Milk Thistle extract+20 mg flaxseed extract+750 mg oat fiber extract) on top of their common diet, 60 day in advance of their surveillance screening colonoscopy. Urinary lignans assessed phytoestrogens intake by the diet and ADI compliance. ERβ, ERα (mRNA, Elisa, immunohistochemistry), TUNEL, caspase-3 and Ki-67 were assessed in the non-adenomatous (normal appearing) mucosa from non-recurrent and recurrent patients. Study power: 80%, type 1 error: 0.05. Two-sided Wilcoxon rank sum test weighted efficacy and Spearman correlation the association to the diet or allocated supplements.

Phytoestrogen intake from the common diet was unchanged in placebo, resulting unrelated to ERs expression. ERα was higher in the non-adenomatous mucosa of non-recurrent (P=0.02) and recurrent patients (P=0.04) in placebo only. ADI increased ERβ protein content (P=0.04). Particularly in recurrent patients, ADI increased ERβ protein content (P=0.04), TUNEL (P=0.04) and caspase-3 demonstrating the functional link between ERβ and apoptosis. directly correlating with co-localized caspase-3 (P<0.004), and inversely with Ki-67 (P=0.04).

This randomized, double-blind and placebo controlled study proved the short-term exposure to ADI, to positively impact on the ERβ-driven apoptotic control of colon epithelial turnover, by increasing ERβ content in the non-adenomatous mucosa of SAP patients prone to polyp recurrence. Similarly to the Apc^{Min/+} altered proliferative to apoptotic ratio in the non-adenomatous mucosa, the normal mucosa of a somatic APC mutated intestinal environment is prone to polyp development and recurrence because of a decreased, or deficient, ERβ expression. Irrespective of polyp recurrence at the screening colonoscopy, the ERβ-dependent apoptosis can be recovered by supplementing phytoestrogens and qualified insoluble fibers, selectively active on the ERβ. ADI did not increase the total ERα, displaying a safe chemopreventive potential for the dietary supplementation of human intestinal polyposis, not basically achieved with the common diet. Notably, the functional biomarkers for the apoptotic control of cell proliferation TUNEL and cleaved caspase-3 directly correlated with the induced ERβ, whereas the proliferative biomarker Ki-67 resulted inversely correlated, and dependent upon the active supplementation and the induced, increased ERβ protein content.

The dietary approach of the study required to prove the active supplementation as effective on ERβ-controlled apoptosis, when added on the patient's common dietary habits. The diet is among the facilitating factors for the silencing of transcriptional programs for the apoptotic control of cell proliferation. Therefore, the basic assessment of urinary enterodiol and enterolactone is a valid conceptual alternative to entry colonoscopy, due to the fact that urinary and plasma lignans are recognized dose-dependent markers for phytoestrogens intake [Penttinen P. et al., 2007] and lignans have been related to a reduced risk for CRC [Kuijsten A. et al., 2006]. Despite their high intra- and inter-individual variability, the randomized, double blind and placebo controlled design, and most importantly the overall content of the baseline urinary lignans did not differ between groups, confirming the study population to be similarly contributed in phytoestrogens from the common diet. The placebo left substantially unchanged those contents over the study period, thus reflecting no inference on the basic phytoestrogen intake. The lacking relationship between the baseline urinary phytoestrogen intake and ERs expression at completion of the 60 day exposure, adds relevant dietary information not otherwise provided with a T0 colonoscopy. Worth noting that the patients lived in Puglia, a southern European region where the availability of phytoestrogens and fibers from fruit, vegetable and cereals, and olive oil consumption is among the highest. Despite the epidemiological evidence of a reduced risk for gastrointestinal cancers because of these dietary habits, the results suggest that a specifically ERβ-targeted dietary supplementation on top of the common diet is needed to effectively impact the balance between proliferation and apoptosis in colon epithelial turnover. HRT or other phytoestrogens, ASA or NSAIDs were not allowed before and during the study period, strengthening the observed changes in ERβ and in ERβ-driven apoptotic biomarkers to depend on ADI. The higher T30 and T60 urinary lignans clearly demonstrated compliance to the active supplement. Notably, the ERβ increase and its correlation to functional biomarkers of apoptosis by treatment were encountered in ADI only, differentiating the active supplementation from the common diet (i.e. placebo), not presenting such relationships either at baseline or at end of study. Relevant to the results, patients administered daily low-dose ASA, NSAIDs, or prescribed those drugs for any reason were not eligible or violated the study protocol, making our patients naïve from any anti-inflammatory control of proliferative and apoptotic biomarkers. Comparing the study herein described with the study performed by Limburg P J et al. [ref. cit.], the placebo was similarly composed of maltodextrins, but no effect on either ERβ or apoptic activity was observed in placebo patients, for whom basic phytoestrogen intake was instead known.

Single ingredients composing the ADI blend have a sounded track of effective chemoprevention on carcinogenesis. Beyond its specific ERβ agonism, silymarin exerts an anti-5-lipoxygenase (LOX) and anti-COX₂ effect. A strong positive correlation has been recently established between 5-LOX overexpression and the appearance of typical high-risk factors for malignant transformation of polyps, such as histological epithelial localization, increased polyp size, villous and tubulovillous adenomas, high grade of intraepithelial neoplasia, and patient age [Wasilewicz M P et al., 2010]. Both inflammatory enzymes are up-regulated in colon carcinogenesis and involved in silencing of apoptosis. Without any apparent toxicity, the feeding of polyphenols from silymarin, silybinin, suppressed the tumor growth of the human SW480 CRC, implanted in nu/nu mice [Kaur M et al., 2010]. The inhibitory activity was associated with strong anti-proliferative (β-catenin, c-Myc and cyclin D1 suppression) and pro-apoptotic effects. Lignans, particularly SDG, exert similar activity in several human colon cancer cells and are easily metabolized and absorbed in the colon [Kuijsten A. et al., 2006]. Insoluble and indigestible fibers, particularly those documented as indigestible for their lignin content, are documented absorbents of carcinogens into the intestinal lumen. The degradation of lignin to enterolignans by the human intestinal microbiota could further provide a delayed lignan release [Mueller S. et al., 2004].

Notwithstanding the association of the three component clearly qualified and in a well-established ratio, as in the compositions according to the invention, has shown with just a two month supplementation in SAP patients prone to polyp recurrence, at intermediate risk for CRC, a remarkable effect in the induction and/or maintenance of ERβ in the non-adenomatous mucosa of non-recurrent and recurrent patients, respectively. In addition, compositions of the invention counterbalance the prevailing ERα-dependent cell proliferation discovered in the non-adenomatous mucosa of SAP patients prone to polyp recurrence.

Therefore, the compositions according to the invention can be usefully employed, either as nutraceuticals (e.g. food supplements, food for special medical purpose or medical food) or pharmaceutical products, in the treatment of patients affected by colon adenomas, diagnosed through colonoscopic examination, with the aim of reducing polyp number and volume (i.e. polyp regression), of preventing their progression to advanced adenomas and, possibly, to adenocarcinoma. Compositions according to the invention can also be employed to prevent or reduce the development of new polyps (i.e. polyp recurrence) and to prevent their progression to an advanced form, and possibly to adenocarcinoma, in polypectomised patients currently recruited to screening colonoscopy program, in the period between scheduled screening colonoscopies.

### Experimental Part

### Materials and Methods

60 eligible SAP patients, men and menopausal women aged at least 50 years, were selected among those polypectomized since 2003 at Gastroenterology Unit of University Hospital of Bari (Italy), found affected by multiple polyps <10 mm in diameter or one-two adenomas ≧10 mm, and/or with a grade of dysplasia to classify them at intermediate risk for CRC. Menopause was defined as the absence of menstruations since 2 years from enrollment. In compliance with guidelines (European Guidelines for quality assurance in colorectal cancer screening and diagnosis. doi:10.2772/15379. 2010), they should have been regularly screened by colonoscopy every 3-5 years for the follow-up of polyp recurrence and progression to CRC. They should not have been previously or concomitantly prescribed HRT, other supplemented phytoestrogens, ASA or NSAIDS. N=600 SAP patients were interviewed for study participation, but the eligibility requirements restricted the enrolled population to 60 patients. They were sequentially 1:1 randomly allocated to placebo (PL) or the active dietary intervention (ADI) at baseline (T0), sixty days in advance of the surveillance screening colonoscopy (T60). The 60 day supplementation was expected to cover the occurrence of approximately eight complete colon epithelial turnovers. Investigational supplements were orally administered twice a day, on top of the common diet. N=50/60 patients constituted the final Per Protocol Population (PPP) for end-point assessment in non-adenomatous (commonly regarded as “normal”) mucosa biopsies, collected during the T60 screening colonoscopy, coincident with the end of the 60 day dietary supplementation. Inclusion criteria were: hemoglobin≧12.0 g/dL; platelets≧120,000/mm³; INR≦1.5; AST or ALT≦1.5 times the upper limit of normal values (ULN); Alkaline Phosphatase≦1.5 times ULN; Bilirubin≦1.5 times ULN; BUN≦5 40 mg/dL; normal blood pressure or controlled hypertension. Exclusion criteria were: chronic inflammatory intestinal disease, intestinal and/or extra-intestinal malignant neoplasms, acute or chronic renal disease, anemia, coagulation disorders, BMI>30. Anti-cancer treatment within 6 months from enrollment, systemic corticosteroids; anticoagulants or anti-platelet aggregation agents, antibiotics within 30 days from enrollment were all non-eligibility criteria. Computer-generation of sequentially randomized 1:1 allocation list, study monitoring, database acquisition, randomization key opening and statistical analysis were fully contracted to an independent Clinical Research Organization (Medical Trial Analysis, MTA, Ferrara, Italy). The study was approved by the Ethics Committee of the University Hospital of Bari [N. 1410]. All patients signed the informed consent, in accordance with the Helsinki Declaration, revision 1983. The study was registered at Clinical Trial.gov (ID: NCT01402648).

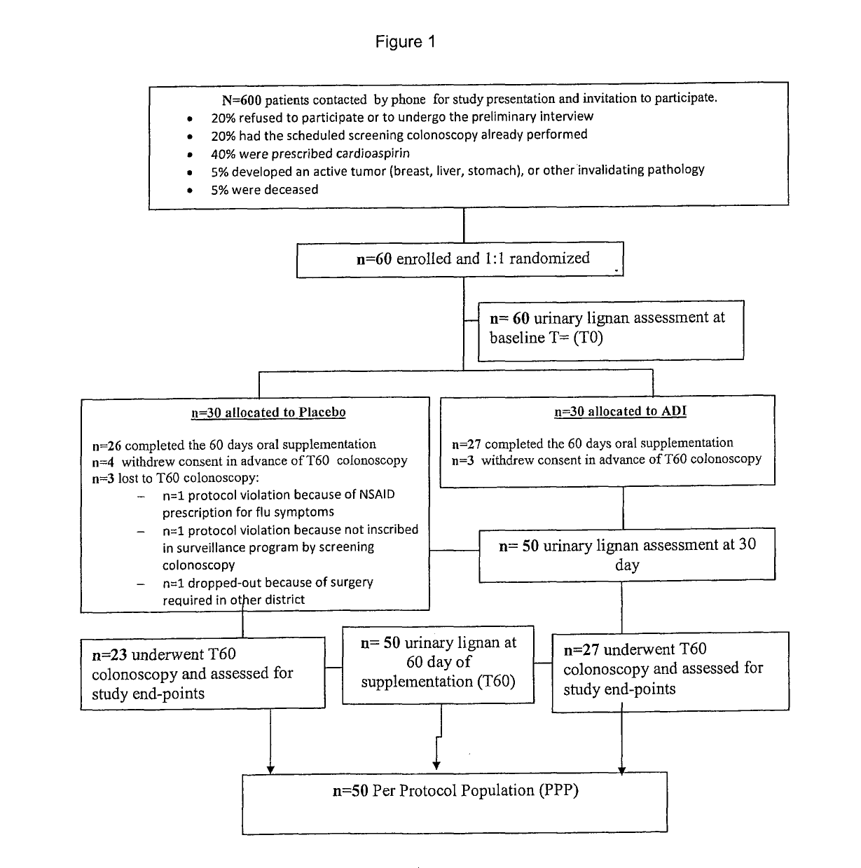

The study design and flow chart of the clinical trial on SAP patients is reported in FIG. 1.

Diet and T60 colonoscopy. To provide a real insight of their dietary habits, and of the relationship between end-points and the common diet or the allocated supplements, the enrolled patients were not previously or concomitantly suggested a recommended diet. Phytoestrogens contribution from the common diet was assessed on spot urine samples at baseline (T0). On a formulative basis, ADI only could add phytoestrogens to the patient's basic intake. The additional contribution from allocated supplements was verified at 30 (T30) and 60 (T60) days. Urinary enterodiol (ED, ng/mL) and enterolactone (EL, ng/mL) were the selected markers for phytoestrogen (lignan) intake. This approach documented compliance to ADI, and discriminated any change in proliferative and apoptotic biomarkers assessed at T60 as differently related to the basic phytoestrogen intake, or to the ADI. Although a high intra- and inter-individual variability in urinary or plasma phytoestrogens is reported in literature, the randomized, double blind and placebo controlled study design allowed the T0 and T60 urinary ED+EL assessment to substitute for T0 colonoscopy. A repeat colonoscopy within 8 weeks was judged an unethical procedure for a proof of concept study in our SAP patients, for whom the current standard of care remains the surveillance program by screening colonoscopy.

Biopsy collection and end points. Five days in advance of T60 colonoscopy, patients refrained from fresh and cooked fruit and vegetable intake. Bowel cleansing was achieved by PEG 4000 oral administration (1120 g/4 L water solution). Numbers of encountered polyps were recorded. Small polyps (≦0.5 cm) were topically electro-coagulated, whereas polyps (>0.5 cm) were submitted to histological assessment. In all patients, irrespective of being currently found recurrent or non-recurrent, n=8 biopsies/patient were collected from the sigmoidal, non-adenomatous colon mucosa. Biopsies were assessed for: ERβ and ERα mRNA, protein (Elisa) and number of immunostained cells (% of immunostained cells over the total number of cells counted per field: % IHC); TUNEL (% IHC), caspase-3 (% IHC), Ki-67 (% IHC), and comparison (mean, median, % IHC) between study groups. Safety was assessed by unchanged hematochemistry over the study period, and no overall induction of ERα expression by ADI.

ERs mRNA. Total RNA was extracted using RNA easy mini kit (Qiagen), according to the manufacturer's instruction. RNA concentration and quality were assessed by spectrophotometric readings at 260 and 280 nm. ERβ and ERα cDNA were generated by Reverse Transcription of 0.5 μg of total RNA in a 20-μl reaction volume (iScript Select cDNA Synthesis Kit, Biorad), as per manufacturer's instruction. 0.5 μL of β-actin cDNA served as internal control. Two rounds of amplification (PCR1 and PCR-2) were performed. PCR-1 was performed in 50 μl final volume (iTaq DNA Polymerase Kit-Biorad) containing 2 μl cDNA and 40 pmol of outer primers (26) through a denaturation step (95° C. for 3 min), 30 cycles (94° C. for 30 sec, 55° C. for 30 sec, 72° C. for 30 sec) and a final primer extension step (72° C. for 10 min). For PCR-2, PCR-1-derived ERβ and ERα were tenfold diluted, and 3 μl amplified with 40 pmol of inner primers as per PCR1 conditions. DNA fragments were separated on a 2% agarose gel stained with ethidium bromide and size assessed by comparison with 100 by DNA marker using Molecular Imager ChemiDOC XRS+ (Biorad). After normalization to the fluorescent β-actin PCR band intensity, ERβ and ERα mRNA were expressed in Arbitrary Units of Fluorescence (AUF).

ERs ELISA. Biopsies were homogenized in lysis buffer (100 mmol/L Tris-HCl (pH 7.5), 300 mmol/L NaCl, 4 mmol/L EDTA, 2% NP40, 0.5% Na deoxycholate, 1 mmol/L sodium orthovanadate) and a protease inhibitor cocktail (Roche) was added. Surnatants were collected (centrifugation 13000× for 25 min) and the Bradford method (Biorad) applied for protein content. ERs were assessed by the QuantiSir specific gene knockdown quantification kit (Epigentek, Brooklyn, N.Y.), following manufacturer's instructions. Protein content was normalized to GAPDH and expressed in Optical Densities (OD) by computer-assisted densitometry. Protein content was normalized to GAPDH and expressed in Optical Densities (OD) by computer-assisted densitometry.

Immunohistochemistry (IHC) and colocalization of ERβ and Caspase-3. Formalin-fixed, 4-μm thick paraffin embedded sections were processed for general histology, and classified as normal, hyperplastic or dysplastic by a blinded gastrointestinal pathologist (DP). Monoclonal antibodies used: ERα (F-10-sc-8002, Santa Cruz, Calif., USA), ERβ (NCL-ERβ, Novocastra Menarini, Milano), TUNEL (in situ Cell Death Detection Kit, Roche), cleaved caspase-3, and Ki-67 (clone MIB-1, DAKO). Counted in blinded fashion by two independent observers (MPS, ST) at ×400 magnification (Leica TSC SP2 confocal laser scanning microscope), immunostained cells were expressed as % over the total cell numbers in ten well oriented crypts and villi. For co-localization assessment, the same primary antibodies as per IHC were used. The antigen was retrieved under shaking conditions in TBS buffer with TWEEN 0.025% and microwave irradiation in citric buffer at pH 6.0. Slides were incubated for 1 hour at room temperature in 1% BSA blocking solution and then overnight dipped at 4° C. in a mixture of the two primary antibodies (ERβ 1:50, Caspase-3 1:30). A mixture of the secondary antibodies Alexa 488 fluorescent-conjugated anti-mouse and Alexa 555 fluorescent-conjugated Goat anti-Rabbit (Invitrogen) in PBS was used for detection. TOPRO-3 (Invitrogen) served the nuclear counterstaining.

UPLC-MS/MS. Concentrations of ED and EL (ng/mL) in human urine spot samples were extracted according to Grace et al. [Grace P B et al., 2007] (mean, SD, CV and RE), using a Waters Quattro Premier Mass Spectrometer and Waters Acquity equipments. In addition to samples, all analytical batches contained two full calibration lines (Quality Controls, QCs) prepared in PBS, corresponding to five different concentrations over the ranges of 0.5 to 2000 ng/mL. Duplicate QCs in PBS at three concentrations covering the calibrated range, and duplicate QCs of human urine whose endogenous ED and EL concentrations had been characterized previously, were also extracted. In batches containing diluted samples, duplicate dilution QCs were prepared by diluting a concentrated spiked PBS sample with additional PBS to the same extent as the study samples. For each set of samples, defined limits for the Relative Errors in the analysis of the standards had to be met before accepting the results.

Investigational dietary supplements and double-blind design. ADI (Eviendep®, CM&D Pharma Limited, UK) is composed of 750 mg insoluble and indigestible oat fiber (69% cellulose, 25% hemicellulose and 6% lignin), 20 mg flaxseed dry extract (titered at 40% secoisolariciresinoldiglucoside, SDG), and 175 mg Milk Thistle extract (titered at 70% silymarin and 30% silybinin by HLPC). PL contained 910 mg maltodextrins, and 100% excipients as per ADI. ADI and PL were provided by the Sponsor (CM&D Pharma Limited, UK). Boxes and sachets were undistinguishable between PL and ADI, and labeled with the protocol code and the allocated sequential number. The 5 g powder sachet had to be dissolved in half glass water and swallowed twice a day. One box/patient of PL or ADI (containing 60 sachets each) were provided at T0 and T30, dispensed to the Clinical Investigators by the Hospital Pharmacy according to the sequential randomized 1:1 allocation list. This assured the double blindness of investigators and patients.

Statistical analysis. Data to refer to on selected biomarkers in SAP, non-adenomatous mucosa are quite scanty. To assess non inferiority, we referred to data available from healthy volunteers and SAP adenomatous tissue [Di Leo A. et al., 2008]. Sample size estimation was based on the assumption of an equivalence margin between allocated supplements of 0.05, an actual difference (mean_{ADI}-mean_{PL}) of 1, SD_{ADI}=2 and SD_{PL}=2. The study power was 80% and Type 1 error 0.05 (two-tails). The non-parametric, two sided Wilcoxon Rank Sums test weighted ERs and biomarkers expression in study groups. ANOVA compared demographic data, urinary phytoestrogens, and study groups distribution around the ERs cut-off value (PPP average median value). In this regard, ERs protein content was classified as lower, equal or higher the PPP cut-off value. Spearman correlation assessed relationships among ERs and the other biomarkers by the common diet and the allocated supplements. A SAS version 8.2 (Statistical Analysis Software, Cary, N.C.) was used.

Results

Table 1 reports baseline demographics for PPP and comparison between study groups, showing good comparability.

TABLE 1
Baseline Demographics (PPP) and comparison between the study groups

| DEMOGRAPHIC | | | PL | ADI | PPP | |
|---|---|---|---|---|---|---|
| DATA | | | (n = 23) | (n = 27) | (n = 50) | P* |
| Sex | Male | n (%) | 17 (73.9%) | 19 (70.4%) | 36 (72.0%) | 1.00 |
| | Female | n (%) | 6 (26.1%) | 8 (29.6%) | 14 (28.0%) | |
| Age | mean ± sd | | 61.6 ± 5.2 | 62.6 ± 6.5 | 62.1 ± 5.9 | 0.56 |
| (years) | median | | 60.0 | 63.0 | 61.0 | |
| Weight | mean ± sd | | 75.5 ± 13.8 | 78.8 ± 12.6 | 77.1 ± 13.1 | 0.32 |
| (kg) | median | | 80.0 | 80.0 | 80.0 | |
| Height | mean ± sd | | 169.5 ± 9.0 | 170.6 ± 9.5 | 170.1 ± 9.2 | 0.67 |
| (cm) | median | | 172.0 | 172.0 | 172.0 | |
| Season | Spring | n (%) | 2 (8.7%) | 5 (18.5%) | 7 (14.0%) | 0.29 |
| of | Summer | n (%) | 2 (8.7%) | 6 (22.2%) | 8 (16.0%) | |
| Enrol- | Autumn | n (%) | 15 (65.2%) | 11 (40.8%) | 26 (52.0%) | |
| ment | Winter | n (%) | 4 (17.4%) | 5 (18.5%) | 9 (18.0%) | |

*ANOVA, t test. Season of enrollment did not differentiate the study groups (P = 0.29). This evidence excludes that different availabilities and/or phytoestrogen intake contributed from fruits, vegetables and cereals in the common diet influenced the outcomes.

Table 2 reports urinary lignan excretions (ED+EL, ED, EL; ng/mL) at T0, T30, T60, and changes (Δ) in study groups, at different timepoints.

TABLE 2
Urinary lignans (ng/ml) at different timepoints

| | Placebo | ADI | |
|---|---|---|---|
| | (n = 23) | (n = 27) | P |
| ED + EL_{T0} | 697.8 ± 963.3 | 410.3 ± 336.9 | 0.27 |
| ED + EL_{T30} | 472.4 ± 475.5 | 3300.1 ± 1535.5 | <.001 |
| ED + EL_{T60} | 116.0 ± 235.2 | 327.5 ± 313.1 | <.001 |
| Δ ED + EL_{T30-T0} | −251.2 ± 1085.9 | 2889.8 ± 1445.0 | <.001 |
| Δ ED + EL_{T60-T0} | −393.9 ± 415.0 | −82.8 ± 380.7 | <.02 |
| ED_{T0} | 117.3 ± 135.6 | 51.8 ± 55.0 | 0.06 |
| ED_{T30} | 65.6 ± 63.1 | 1084.6 ± 1066.9 | <.001 |
| ED_{T60} | 17.2 ± 48.5 | 77.2 ± 75.1 | <.001 |
| Δ ED_{T30-T0} | −57.1 ± 156.4 | 1032.8 ± 1041.6 | <.001 |
| Δ ED_{T60-T0} | −89.9 ± 105.6 | 25.4 ± 96.7 | <.001 |
| EL_{T0} | 580.5 ± 853.3 | 358.5 ± 335.0 | 0.35 |
| EL_{T30} | 391.3 ± 440.8 | 2215.5 ± 1366.2 | <.001 |
| EL_{T60} | 81.6 ± 177.4 | 250.3 ± 275.2 | <.001 |
| Δ EL_{T30-T0} | −189.2 ± 931.1 | 1857.0 ± 1199.1 | <.001 |
| Δ EL_{T60-T0} | −498.8 ± 861.9 | −108.2 ± 320.1 | <.01 |

Urinary excretion (mean ± sd; ng/mL) of ED + EL, ED, EL, and Δ at T0, T30 and T60 of oral supplementation.
Grey rows: urinary lignans from the regular diet (T0).
In PL group: at T30, no differences in urinary lignans were observed, confirming PL as unable to provide additional phytoestrogens on top of the common diet [ED + EL_{T30vsT0} (P = 0.17); ED_{T30vsT0} (P = 0.11); EL_{T30vsT0} (P = 0.35), at T60: the significant differences are due to bowel preparation procedures in advance of the screening colonoscopy [ED + EL_{T60vsT0} (P = 0.004)]; ED_{T60vsT0} (P = 0.004); EL_{T60vsT0} (P = 0.007)].
In ADI group: phytoestrogen supplementation is clear at T30 and T60 also, partly counterbalancing the effects of bowel preparations [ED + EL_{T30vsT0} (P < 0.0001); ED_{T30vsT0} (P < 0.0001); EL_{T30vsT0} (P < 0.0001). ED + EL_{T60vsT0} (P = 0.35); ED_{T60vsT0} (P = 0.16); EL_{T60vsT0} (P = 0.20)].

Total urinary ED+EL_{T0}, ED_{T0} and EL_{T0} in PL and ADI groups confirmed a similar phytoestrogen contribution from the common diet, irrespective of its composition and seasonality. In PL group, no differences in T30 urinary lignans were observed, confirming PL as unable to provide additional phytoestrogens on top of the common diet. The expected fall in T60 urinary lignans reflected bowel preparation and cleansing in advance of the colonoscopy, halting the intestinal microbiota involved in the metabolisation of dietary lignans before mucosal absorption. Spearman correlation ran by T0 urinary lignans assessed the influence on ERs, caspase-3 and Ki-67 as related to the basic phytoestrogen intake (diet), and left unchanged by PL supplementation. ED_{T0} or EL_{T0} did not correlate with ERβ or ERα (mRNA, protein and % ERs immunostained cells), nor they did with the other apoptotic and proliferative biomarkers. This result suggests no inference from the differently ERs-targeted lignans introduced with the diet on either ERα or ERβ expression. Nonetheless, the total ED+EL_{T0} directly correlated with caspase-3 (% IHC, r=0.490, P=0.02) and ERα (% IHC, r=0.501, P=0.01), supporting basic phytoestrogen intake with the common diet to relate with colon epithelial turnover, in terms of cell apoptosis (caspase-3) and cell proliferation (ERα). In ADI group, urinary ED_{T30,T60} (P<0.001), EL_{T30,T60} (P<0.001), total ED+EL_{T30,T60} (P<0.001), and Δ_{T30-T0}, Δ_{T60-T30} increased (P<0.001 and P=0.01, respectively) demonstrating active phytoestrogen supplementation on top of the common diet, and compliance to ADI. Urinary ED+EL_{T60} remained significantly higher in ADI (P<0.001), despite the bowel cleansing procedure.

Table 3 reports the ERβ and ERα mean protein content and the median cut-off value assessed in non-adenomatous biopsies at T60.

TABLE 3
ERs protein content and patients distribution around the ERs cut-off value

| | | PL | ADI | PPP | |
|---|---|---|---|---|---|
| | | (n = 23) | (n = 27) | (n = 50) | P |
| ERβ | mean ± sd | 0.77 ± 0.1 | 0.82 ± 0.1 | 0.8 ± 0.1 | 0.04 |
| | Median (cut-off | 0.76 | 0.84 | 0.82 | |
| | value) | | | | |
| | ≦0.82 | 15 (65.22%) | 10 (37.04%) | 25 (50.0%) | 0.05 |
| | >0.82 | 8 (34.78%) | 17 (62.96%) | 25 (50.0%) | |
| ERα | mean ± sd | 0.51 ± 0.1 | 0.49 ± 0.1 | 0.50 ± 0.1 | 0.44 |
| | Median (cut-off | 0.50 | 0.45 | 0.47 | |
| | value) | | | | |
| | ≦0.47 | 8 (34.8%) | 18 (66.7%) | 26 (52.0%) | 0.02 |
| | >0.47 | 15 (65.2%) | 9 (33.3%) | 24 (48.0%) | |

ERβ and ERα [mean ± sd of protein content in Elisa (OD); median] in the non-adenomatous mucosa and patients' distribution around the median cut-off value.

ADI increased the ERβ protein content (P=0.04), leaving unchanged the ERα (P=0.44). The whole PPP (n=50) had a ERβ cut-off value of 0.82 OD. The null hypothesis predicted the patients in the two groups to equally distribute (50:50) around the cut-off, to negate any effect from ADI. Instead, 62.96% of ADI patients vs 34.78% in PL (P=0.05) fell above the ERβ cut-off. The ERα median cut-off was 0.47 OD. The null hypothesis predicted the patients in the two groups to distribute 52% below and 48% above the cut-off, to negate any effect from ADI. Instead, only 33.3% of ADI patients fell above the ERα cut-off vs 65.2% in PL (P=0.02). These results demonstrate the ADI to positively impact ERβ expression, counterbalancing the prevailing ERα phenotype of the non-adenomatous mucosa of SAP patients prone to polyp recurrence. Notably, no overall changes in ERα protein and mRNA content, or ERα% were observed in ADI (P=0.6). The ADI-induced increase in ERβ content with no ERα induction, provided the proof of concept of ADI as a safe, selected blend of specific dietary ERβ inducers.

Table 4 reports ERs, Ki-67, TUNEL and caspase-3 (means±sd) expression in the non-adenomatous mucosa of patients found free of polyps (i.e. currently non-recurrent) or with one/multiple polyps (i.e. currently recurrent) at T60 screening colonoscopy, and requiring in situ electrocoagulation (φ≦0.5 cm) or polypectomy for histological assessment (P+H, φ>0.5 cm).

TABLE 4
Biomarkers in the non-adenomatous mucosa of non-recurrent and recurrent patients

| | Non-recurrent patients | | Recurrent patients | |
|---|---|---|---|---|
| Biomarker | PLACEBO | ADI | PLACEBO | ADI |
| mean ± sd | N = 16/23 | N = 9/27 | N = 6/23 | N = 14/27 ^{(a)} |
| ERα mRNA | 0.159 ± 0.32 | 0.295 ± 0.26 | 0.097 ± 0.11 | 0.209 ± 0.26 |
| ERα LI | 16.75 ± 8.86 | 15.33 ± 9.76 | 29.50 ± 10.23^{(a)} | 18.78 ± 12.01 |
| ERα protein | 0.532 ± 0.11 | 0.429 ± 0.06⁻ | 0.485 ± 0.11 | 0.518 ± 0.14^{(c)} |
| Ki-67 LI | 45.31 ± 11.29 | 55.33 ± 20.41 | 43.66 ± 10.31 | 53.14 ± 21.74 |
| ERβ mRNA | 1.105 ± 1.07 | 2.278 ± 1.19^{-(c)} | 1.471 ± 0.61 | 0.730 ± 0.98^{(b)} |
| ERβ LI | 34.875 ± 16.57 | 47.533 ± 15.48 | 38.83 ± 12.84 | 35.14 ± 18.80^{(b)} |
| ERβ protein | 0.773 ± 0.13 | 0.806 ± 0.13^{-(c)} | 0.763 ± 0.05 | 0.807 ± 0.14^{-(d)} |
| Caspase-3 LI | 29.53 ± 9.13 | 39.44 ± 15.60^{(c)} | 31.00 ± 3.8 | 29.09 ± 12.79 |
| TUNEL LI | 31.375 ± 12.93 | 43.78 ± 17.24^{(c)} | 32.00 ± 10.54 | 31.93 ± 13.3⁻ |

Statistical analyses: ^{(a)}ADI vs PL; ^{(c)}Non-recurrent: ADI vs PL; ^{(d)}Recurrent: ADI vs PL; ^{(a)}within PL group: Recurrent vs Non-recurrent; ^{(b)}within ADI group: Recurrent vs Non-recurrent

A higher percentage of ADI patients was found currently recurrent (P=0.03). Whereas adenoma development in SAP is a long-term process, our short-term exposure study (60 days) excludes any inference on adenoma recurrence, except on biomarkers' expression. In non-recurrent patients, the non-adenomatous mucosa of ADI group showed an increase in ERβ mRNA (P=0.05), ERβ protein (P=0.04), % TUNEL immunostained cells (P=0.05), and a lower content in ERα protein (P=0.02). Irrespective of polyp recurrence encountered at screening colonoscopy, and similarly to our previous observation in the Apc^{Min+/-} mice, these results demonstrated a prevailing ERα proliferative behaviour in the non-adenomatous mucosa of SAP patients (as per PL group). The ADI counterbalanced this behaviour increasing the expression of ERβ and TUNEL, that is: the apoptosis-related biomarkers. In the non-adenomatous mucosa of recurrent patients, the % ERα cells significantly increased in PL group only (P=0.05 vs non-recurrent PL, and recurrent ADI). This finding was not confirmed in the non-adenomatous mucosa of ADI recurrent patients, where an unchanged % ERα cells vs either non-recurrent PL or non-recurrent ADI patients was found. Nonetheless, an increased ERα protein content (P=0.05) suggested the presence of a subset of cells with an increased ERα content/cell, a finding compatible with the higher adenoma multiplicity encountered at screening colonoscopy in recurrent ADI patients. In ADI only, the overall ERβ protein content increased (P=0.05 vs recurrent PL patients), despite the decreased ERβ mRNA (vs non-recurrent ADI patients, P<0.004). The higher ERβ content could suggest a more efficient protein synthesis and/or a delayed protein degradation by ADI. Spearman correlation ran by allocated supplement showed in ADI group only: % ERβ cells directly correlated with % TUNEL cells (r=0.406, P=0.03 vs r=0.379, P=0.07 in PL), % caspase-3 cells (r=0.529, P<0.004 vs r=0.134, P=0.55 in PL), and ERβ protein inversely correlated with the % Ki-67 cells (r=−0.403, P=0.04 vs r=0.009, P=0.97 in PL).

Overall, these results confirm the ADI as effective in the induction and/or maintenance of ERβ in the currently non-adenomatous mucosa of non-recurrent and recurrent patients, respectively. Differently from the common diet—mirrored in PL group—our results support the ADI to counterbalance the prevailing ERα-dependent cell proliferation discovered in the non-adenomatous mucosa of SAP patients prone to polyp recurrence.

### REFERENCES

Vogelstein B, Fearon E R, Hamilton S R, Kern S E, Preisinger A C, Leppert M, et al. Genetic alterations during colorectal-tumour development. N Engl J Med 1988; 319:525-32.
Duthie S J. Epigenetic modifications and human pathologies: cancer and CVD. Proc Nutr Soc 2011; 70:47-56.
Lund E K et al. Recent advances in understanding the role of diet and obesity in the development of colorectal cancer. Proc Nutr Soc 2011; 70:194-204.
Gingras D, Béliveau R. Colorectal cancer prevention through dietary and lifestyle modification. Cancer Microenviron 2011; 4:133-39.
Woodson K, Lanza E, Tangrea J A, Albert P S, Slattery M, Pinsky J, et al. Hormone Replacement Therapy and Colorectal Adenoma Recurrence Among Women in the Polyp Prevention Trial. J Natl Canc Inst 2001; 93:1799-1805.
Bardin A, Boulle N, Lazennec G, Vignon F, Pujol P. Loss of ERβ expression as a common step in estrogen-dependent tumor progression. Endocrine-Related Cancer 2004; 11:537-51.
Chan A T, Arber N, Burn J, Chia W K, Elwood P, Hull M A, Logan R F, Rothwell P M; Schrör K; and Baron J. Aspirin in the chemoprevention of colorectal neoplasia. Cancer Prey Res 2012: 164-178.
Di Leo A, Barone M, WO2009/103788
Barone M, Tanzi S, Lofano K, et al. Dietary-induced ERβ upregulation counteracts intestinal neoplasia development in intact male Apc^{Min/+} mice. Carcinogenesis. 2010; 31:269-274.
Limburg P J, Mahoney M R, Allen Ziegler K L, Sontag S J, Schoen R E, Benya R, et al. Randomised phase II trial of sulindac, artovastatin, and prebiotic dietary fiber for colorectal cancer chemoprevention. Cancer Prey Res 2011; 4:259-69.
Penttinen P, Jaehrling J M, Anastasios E, et al. Diet-derived polyphenol enterolactone is a tissue-specific Estrogen receptor Activator. Endocrinology 2007; 148:4875-4886.
Kuijsten A, Arts I C, Hollman P C, van't Veer P, Kampman E. Plasma enterolignans are associated with lower colorectal adenoma risk. Cancer Epidemiol Biomarkers Prev. 2006; 15:1132-36.
Wasilewicz M P, Kolodziej B, Bojulko T, Kaczmarczyk M, Sulzyc-Bielicka V, Bielicki D, et al. Overexpression of 5-lipoxygenase in sporadic adenomas and a possible new aspect of colon carcinogenesis. Int J Colorectal Dis 2010; 25:1079-85.
Kaur M, Velmurugan B, Tyagi A, Agarwal C, Singh R P, Agarwal R. Silybinin suppresses growth of human colorectal carcinoma SW480 cells in culture and xenograft through down-regulation of beta-catenin-dependent signaling. Neoplasia 2010; 12:415-24.
Mueller S, Simon S, Chae K, et al. Phytoestrogens and their human metabolites show distinct agonistic and antagonistic properties on Estrogen Receptor α (ERα) and ERβ in Human Cells. Toxicol Sci. 2004; 80: 14-25.
Grace P B, Mistry N S, Carter M H, Leathern A J, Teale P. High throughput quantification of phytoestrogens in human urine and serum using liquid chromatography/tandem mass spectrometry (LC-MS/MS). J Chromatogr B Analyt Technol Biomed Life Sci 2007; 853:138-46.
Di Leo A, Barone M, Maiorano E, Tanzi S, Piscitelli, Marangi S, et al. ER-beta expression in large bowel adenomas: implications in colon carcinogenesis. Dig Liver Dis 2008; 40: 260-66.

1. A composition comprising at least one ERβ-targeted phytoestrogens in combination with non-starch, insoluble and indigestible fibers qualified for having not less than 5% lignin content and with lignans qualified for having not less than 40% of secoisolariciresinoldiglucoside content for use for prevention and treatment of sporadic adenomatous polyposis (SAP) and familial adenomatous polyposis (FAP).

2. The composition according to claim 1, wherein the phytoestrogens are selected from the group consisting of silymarin, silybinin, iso-silybinin, silydianin, silychristin, and mixtures thereof.

3. The composition according to claim 1, wherein the phytoestrogen is the Milk Thistle extract.

4. The composition according to claim 1, wherein the phytoestrogen is the Milk Thistle extract titered at not less than 70% silymarin and 30% silybinin.

5. The composition according to claim 1, wherein the insoluble and indigestible fibres, in purified form or as extracts, further comprise cellulose and hemicellulose.

6. The composition according to claim 5, wherein the insoluble and indigestible fibres are selected from the group consisting of extracts of cereals and legumes.

7. The composition according to claim 1, wherein the lignans are selected from the group consisting of extracts of flaxseed, sesame seeds, rapeseeds, soy flour, whole-grain cereals and coffee beans.

8. The composition according to one of the claims from 1 to 7, wherein the components of the compositions are in a ratio by weight of at least of 1:4:0.1 (phytoestrogens as extracts:non-starch insoluble and indigestible fibers:lignans).

9. The composition according to one of the claims from 1 to 7, wherein the components of the compositions are in a ratio by weight of at least of 1:6:0.2 (silymarin:non-starch insoluble and indigestible fibers:lignans).

10. The composition according to one of the claims from 1 to 7, wherein the components of the compositions are in a ratio by weight of at least 1:14:0.4 (silybinin:non-starch insoluble and indigestible fibers:lignans).

11-21. (canceled)

22. A method for preventing and treating sporadic adenomatous polyposis (SAP) and familial adenomatous polyposis (FAP), comprising administering in a subject in need thereof an effective amount of a composition comprising at least one ERβ-targeted phytoestrogens in combination with non-starch, insoluble and indigestible fibers qualified for having not less than 5% lignin content and with lignans qualified for having not less than 40% of secoisolariciresinoldiglucoside content.

23. The method according to claim 22, wherein the the phytoestrogens are selected from the group consisting of silymarin, silybinin, iso-silybinin, silydianin, silychristin, and mixtures thereof.

24. The method according to claim 22, wherein the the phytoestrogen is the Milk Thistle extract.

25. The method according to claim 24, wherein the the phytoestrogen is the Milk Thistle extract titered at not less than 70% silymarin and 30% silybinin.

26. The method according to claim 22, wherein the insoluble and indigestible fibres, in purified form or as extracts, further comprise cellulose and hemicellulose.

27. The method according to claim 26, wherein the insoluble and indigestible fibres are selected from the group consisting of extracts of cereals and legumes.

28. The method according to claim 22, wherein the lignans are selected from the group consisting of extracts of flaxseed, sesame seeds, rapeseeds, soy flour, whole-grain cereals and coffee beans.

29. The method according to claim 22, wherein the components of the composition are in a ratio by weight of at least of 1:4:0.1 (phytoestrogens as extracts:non-starch insoluble and indigestible fibers:lignans).

30. The method according to claim 22, wherein the components of the composition are in a ratio by weight of at least of 1:6:0.2 (silymarin:non-starch insoluble and indigestible fibers:lignans).

31. The method according to claim 22, wherein the components of the composition are in a ratio by weight of at least of 1:14:0.4 (silybinin:non-starch insoluble and indigestible fibers:lignans).

32. The method according to claim 22, wherein the treatment is a secondary chemopreventive treatment of sporadic adenomatous polyposis (SAP) and familial adenomatous polyposis (FAP).
